(19) United States
(12) Patent Application Publication
(10) Pub. No.: US 2019/0219652 A1
(43) Pub. Date: Jul. 18, 2019

(54) SYSTEMS AND METHODS FOR IMAGING USING BLOOD POOL TIME ACTIVITY CURVES

(71) Applicant: General Electric Company, Schenectady, NY (US)

(72) Inventors: Gillan Michael Grimberg, Tel Aviv (IL); Gil Kovalski, Tirat Carmel (IL)

(21) Appl. No.: 15/871,648

(22) Filed: Jan. 15, 2018

(51) Int. Cl. G01R 33/48; A61B 6/00; A61B 6/03; A61B 5/0402; G01R 33/567

(52) U.S. Cl. CPC G01R 33/481; A61B 6/503; A61B 6/4435; A61B 6/4266; G01R 33/5673; A61B 6/5205; A61B 6/5217; A61B 5/0402; A61B 6/037

(57) ABSTRACT

An imaging system is provided that includes at least one detector unit and at least one processor. The at least one detector unit is configured to acquire nuclear medicine (NM) imaging information. The at least one processor operably coupled to the at least one detector unit, and is configured to acquire initial imaging information over an initial time range corresponding to administration of a radiopharmaceutical; acquire cardiac cycle information over the initial time range; determine targeted portions of the initial imaging information based on the cardiac cycle information to provide targeted initial imaging information; generate an activity curve using the targeted portions of the initial imaging information; and reconstruct an image using the activity curve.

### BACKGROUND

The subject matter disclosed herein relates generally to medical imaging systems, and more particularly to radiation detection systems.

In nuclear medicine (NM) imaging, such as single photon emission computed tomography (SPECT) or positron emission tomography (PET) imaging, radiopharmaceuticals are administered internally to a patient. Detectors (e.g., gamma cameras), typically installed on a gantry, capture the radiation emitted by the radiopharmaceuticals and this information is used, by a computer, to form images. The NM images primarily show physiological function of, for example, the patient or a portion of the patient being imaged.

For acquired NM imaging data, blood pool sampling may be employed for calibration of reconstruction processes. However, conventional approaches may provide less than desired accuracy, for example due to heart motion artifacts, spillover (or myocardial wall activity interference), and/or difficulties in accurately determining or identifying a volume of interest for the blood pool.

### BRIEF DESCRIPTION

In accordance with an embodiment, an imaging system is provided that includes at least one detector unit and at least one processor. The at least one detector unit is configured to acquire nuclear medicine (NM) imaging information. The at least one processor operably coupled to the at least one detector unit, and is configured to acquire initial imaging information over an initial time range corresponding to administration of a radiopharmaceutical; acquire cardiac cycle information over the initial time range; determine targeted portions of the initial imaging information based on the cardiac cycle information to provide targeted initial imaging information; generate an activity curve using the targeted portions of the initial imaging information; and reconstruct an image using the activity curve.

In accordance with another embodiment, a nuclear medicine (NM) multi-head imaging is provided that includes a gantry, plural detector units, and at least one processor coupled to at least one of the detector units. The gantry defines a bore configured to accept an object to be imaged. The plural detector units are mounted to the gantry. Each detector unit defines a corresponding view oriented toward a center of the bore, with each detector unit configured to acquire imaging information over a sweep range corresponding to the corresponding view. The at least one processor is configured to acquire initial imaging information, for at least one detector unit, for an initial volume over an initial time range; to acquire general imaging information, for at least some of the detector units, for a general volume over a general time range subsequent to the initial time range, wherein the initial volume is smaller than and included within the general volume, wherein the at least one processor is configured to use different scanning parameters for acquiring the initial imaging information than for the general imaging information; and to reconstruct an image using the initial imaging information and the general imaging information.

In accordance with another embodiment, a method includes acquiring initial imaging information with at least one of plural detector units mounted to the gantry. Each detector unit defines a corresponding view oriented toward a center of the bore, and each detector unit is configured to acquire imaging information over a sweep range corresponding to the corresponding view for each detector unit, wherein the initial imaging information is acquired for an initial volume over an initial time range. The method also includes acquiring general imaging information, with at least some of the detector units, for a general volume over a general time range subsequent to the initial time range. The initial volume is smaller than and included within the general volume. Different scanning parameters are used for acquiring the initial imaging information than for the general imaging information. Further, the method includes reconstructing an image using the initial imaging information and the general imaging information.

### BRIEF DESCRIPTION OF THE DRAWINGS

FIG. 1 provides a schematic view of a nuclear medicine (NM) imaging system according to an embodiment.

FIG. 2 provides a schematic view of a detector arrangement according to an embodiment.

FIG. 3 illustrates an example cardiac wave including associated left ventricle states schematically depicting corresponding volumes of the left ventricle at various portions of the cardiac cycle.

FIG. 4 illustrates an example activity curve in accordance with an embodiment.

FIG. 5 provides a schematic view of a detector having different fields of view for an initial imaging acquisition and a subsequent, general imaging acquisition in accordance with an embodiment.

FIG. 6 shows a schematic view of an imaging system, according to an embodiment.

FIG. 7 shows a flowchart of a method, according to an embodiment.

### DETAILED DESCRIPTION

The foregoing summary, as well as the following detailed description of certain embodiments and claims, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, the terms “system,” “unit,” or “module” may include a hardware and/or software system that operates to perform one or more functions. For example, a module, unit, or system may include a computer processor, controller, or other logic-based device that performs operations based on instructions stored on a tangible and non-transitory computer readable storage medium, such as a computer memory. Alternatively, a module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

As used herein, an element or step recited in the singular and preceded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Various embodiments provide systems and methods for imaging various portions of an object (e.g., a human patient) at different times of an acquisition. Some embodiments provide systems and methods for dynamic myocardial blood pool sampling (e.g., during and end diastolic phase).

In some embodiments, a dynamic and gated acquisition is performed on a patient, and stored as a list file. The list file is then reframed per a dynamic reframing scheme, with each gamma event categorized per a gated bin. Gamma events relevant only to an end diastolic phase may be utilized, so that each utilized frame includes only end diastolic events. The frames are then used as input to a myocardial blood pool sampling method. The sampled data may be normalized, as only a fraction of the events are used per frame. Accordingly, various embodiments provide improved accuracy relative to, for example, approaches that neglect the myocardial cycle.

A technical effect of at least one embodiment includes improved image quality. A technical effect of at least one embodiment includes improved determination of blood pool curves for calibrating imaging or reconstruction processes. A technical effect of at least one embodiment includes reduced heart motion artifacts. A technical effect of at least one embodiment includes reduced spillover or myocardial wall activity interference to an analyzed blood pool. A technical effect of at least one embodiment included improved accuracy and/or reliability in identifying or determining blood pool volumes of interest.

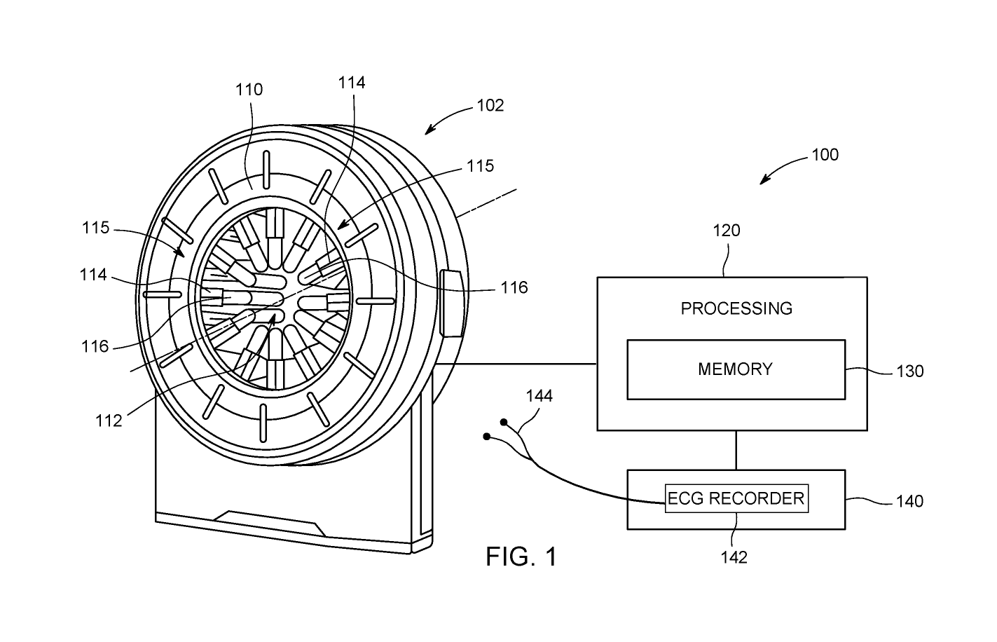

FIG. 1 provides a schematic view of an imaging system 100. The imaging system 100 includes an imaging detector system 102, a cardiac acquisition unit 140, and a processing unit 120. Generally, the imaging detector system 102 acquires imaging information, and the cardiac acquisition unit 140 acquires cardiac cycle information of an object (e.g., human patient) being imaged with the imaging detector system 102. The processing unit 120 acquires information from both the imaging detector system 102 and the cardiac acquisition unit 140, and reconstructs an image using the information. In the illustrated embodiment, the system 100 further includes an ECG (Electrocardiography) recorder 142 for detecting signals from leads 144 attached to the imaged patient.

The imaging detector system 102 in various embodiments includes one or more detector units (e.g., detector unit 115 discussed below) configured to acquire nuclear medicine (NM) imaging information. For example, a radiopharmaceutical may be administered to an object being imaged. Portions of the object being imaged then emit photons. The emissions from different portions of the object vary based on the uptake of the radiopharmaceutical by the corresponding portions. The imaging detector system 102 is used to acquire photon counts which may be used to reconstruct an image of the object.

In the embodiment depicted in FIG. 1, the imaging system 100 is configured as a nuclear medicine (NM) multi-head imaging system. It may be noted that other types or configurations of detectors may be used in other embodiments. Generally, the imaging system 100 depicted in FIG. 1 is configured to acquire imaging information (e.g., photon counts) from an object to be imaged (e.g., a human patient) that has been administered a radiopharmaceutical. The depicted imaging system 100 includes a gantry 110 and a processing unit 120.

The gantry 100 defines a bore 112. The bore 112 is configured to accept an object to be imaged (e.g., a human patient or portion thereof). As seen in FIG. 1, plural detector units 115 are mounted to the gantry 110. In the illustrated embodiment, each detector unit 115 includes an arm 114 and a head 116. The arm 114 is configured to articulate the head 116 radially toward and/or away from a center of the bore 112 (and/or in other directions), and the head 116 includes at least one detector, with the head 116 disposed at a radially inward end of the arm 114 and configured to pivot to provide a range of positions from which imaging information is acquired.

The detector of the head 116, for example, may be a semiconductor detector. For example, a semiconductor detector various embodiments may be constructed using different materials, such as semiconductor materials, including Cadmium Zinc Telluride (CdZnTe), often referred to as CZT, Cadmium Telluride (CdTe), and Silicon (Si), among others. The detector may be configured for use with, for example, nuclear medicine (NM) imaging systems, positron emission tomography (PET) imaging systems, and/or single photon emission computed tomography (SPECT) imaging systems.

In various embodiments, the detector may include an array of pixelated anodes, and may generate different signals depending on the location of where a photon is absorbed in the volume of the detector under a surface if the detector. The volumes of the detector under the pixelated anodes are defined as voxels (not shown). For each pixelated anode, the detector has a corresponding voxel. The absorption of photons by certain voxels corresponding to particular pixelated anodes results in charges generated that may be counted. The counts may be correlated to particular locations and used to reconstruct an image.

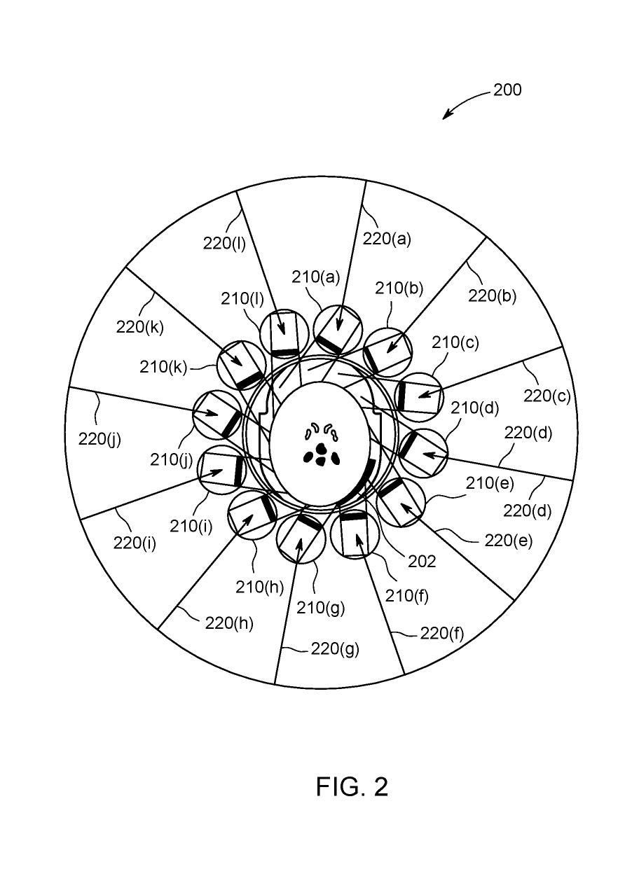

In various embodiments, each detector unit 115 may define a corresponding view that is oriented toward the center of the bore 112. Each detector unit 115 in the illustrated embodiment is configured to acquire imaging information over a sweep range corresponding to the view of the given detector unit. FIG. 2 illustrates a detector arrangement 200 in accordance with various embodiments. The detector units of FIG. 1, for example, may be arranged in accordance with aspects of the detector arrangement 200.

As seen in FIG. 2, the detector arrangement 200 includes detector units 210(a), 210(b), 210(c), 210(d), 210(e), 210(f), 210(g), 210(h), 210(i), 210(j), 210(k), 210(l) disposed about and oriented toward (e.g., a detection or acquisition surface of the detector units, and/or the collimator's FOV (Field Of View), are oriented toward) an object 202 to be imaged in the center of a bore. Each detector unit of the illustrated embodiment defines a corresponding view that may be oriented toward the center of the bore of the detector arrangement 200. The view for each detector unit 210, for example, may be aligned along a central axis of a corresponding arm (e.g., arm 114) of the detector unit 210. In the illustrated embodiment, the detector unit 210(A) defines a corresponding view 220(A), the detector unit 210(B) defines a corresponding view 220(B), the detector unit 210(C) defines a corresponding view 220(C), and so on. The detector units 210 are configured to sweep or pivot (thus sweeping the corresponding FOV's) over a sweep range (or portion thereof) bounded on either side of a line defined by the corresponding view during acquisition of imaging information. Thus, each detector unit 210 may collect information over a range larger than a field of view defined by a stationary detector unit. It may be noted that, generally, the sweeping range that a detector may pivot may be larger than the corresponding view during acquisition. In some cameras, the sweeping range that a detector may pivot may be unlimited (e.g., the detector may pivot a full 360 degrees), while in some embodiments the sweeping range of a detector may be constrained, for example over 180 degrees (from a −90 degree position to a +90 degree position relative to a position oriented toward the center of the bore).

With continued reference to FIG. 1, the depicted cardiac acquisition unit 140 is configured to acquire cardiac cycle information of the object being imaged. For example, the cardiac acquisition unit 140 may include a wearable sensor and/or a non-contact sensor configured to detect cardiac cycle information of a human patient being imaged. In the illustrated embodiment, the cardiac acquisition unit 140 includes an ECG recorder 142 and ECG leads 144. The ECG leads 144 are configured to be attached to the patient being imaged. In various embodiments, an electrocardiogram (ECG) may be acquired. Generally, an ECG provides information regarding the electrical activity of the heart, or a representation of an electrical wave traveling through the heart. The wave causes portions of the heart to contract at different times, with the shape of the wave corresponding to contractions and dilations of the different portions of the heart. For example, the wave may be described as a “PQRST” wave. Generally, the P portion of the wave (or P wave) corresponds to the contraction of the atria (upper chambers) of the heart, with a QRS complex formed by the ventricles (lower chambers of the heart). Following the QRS complex, the T portion of the wave (or T wave) represents electrical recovery of the ventricles.

The depicted processing unit 120 of the example depicted in FIG. 1 is configured to acquire imaging information from the imaging detector system 102, to acquire cardiac cycle information from the cardiac acquisition unit 140, and to reconstruct an image using the acquired imaging information and cardiac cycle information. The imaging information includes initial imaging information that is acquired over an initial time range. In various embodiments, the imaging information also includes general imaging information that is acquired over a time range subsequent to the initial imaging information. (It may be noted that the subsequent time range may commence immediately upon conclusion of the initial time range, may commence after a period of time has elapsed after conclusion of the initial time range, or may commence at a time after the start of the initial time range but before the conclusion of the initial time range, with a portion of the time ranges accordingly overlapping.)

In the illustrated embodiment, the initial imaging information is acquired over an initial time range corresponding to administration of a radiopharmaceutical. For example, the initial time range may be selected to include or correspond to a period of time after the administration of the radiopharmaceutical to a patient but before significant, substantial, or complete uptake of the radiopharmaceutical by one or more target tissues or portions of the patient. In the illustrated embodiment, the initial time range is selected to correspond to a time period during which the radiopharmaceutical is in the blood stream and/or arriving at a target tissue. For example, the initial time range in various embodiments corresponds to a time at which a maximum number of photon counts due to the radiopharmaceutical occurs in a blood pool contained within a heart (e.g., within the left ventricle), which corresponds to arrival of the radiopharmaceutical to the myocardium. In various embodiments, the initial time range defines a range or time window within which the maximum number of photon counts within a blood pool within a ventricle of the heart occurs. The initial time range in various embodiments extends less than 200 seconds after administration of the radiopharmaceutical.

In the illustrated embodiment, the processing unit 120 is configured to determine target portions of the initial imaging information based on the cardiac cycle information to provide targeted initial imaging information. Generally, the cardiac cycle information is registered to the initial imaging information and used to classify the initial imaging information. For example, the cardiac cycle information may be correlated to the initial imaging information (e.g., based on time of acquisition), and imaging information from one or more portions of the cardiac cycle retained as the targeted initial imaging information, with imaging information from other portions of the cardiac cycle discarded or otherwise not included with the targeted initial imaging information. In some embodiments, imaging information corresponding with portions of the cardiac cycle for which the most volume of blood is in a portion of the heart (e.g., left ventricle) may be selected as targeted initial imaging information and used to determine photon counts or uptake of blood for deriving diagnostic parameters. For example, the processing unit 120 may determine the target portions based on a portion of a cardiac cycle corresponding to dilation of a left ventricle. It may be noted that the selected portion(s) of the cardiac cycle may include a range of time defined within a threshold of a maximum or target value, such as a maximum volume of the left ventricle. In some embodiments, the processing unit 120 determines the target portions based on a portion of the cardiac cycle corresponding to an end of diastole.

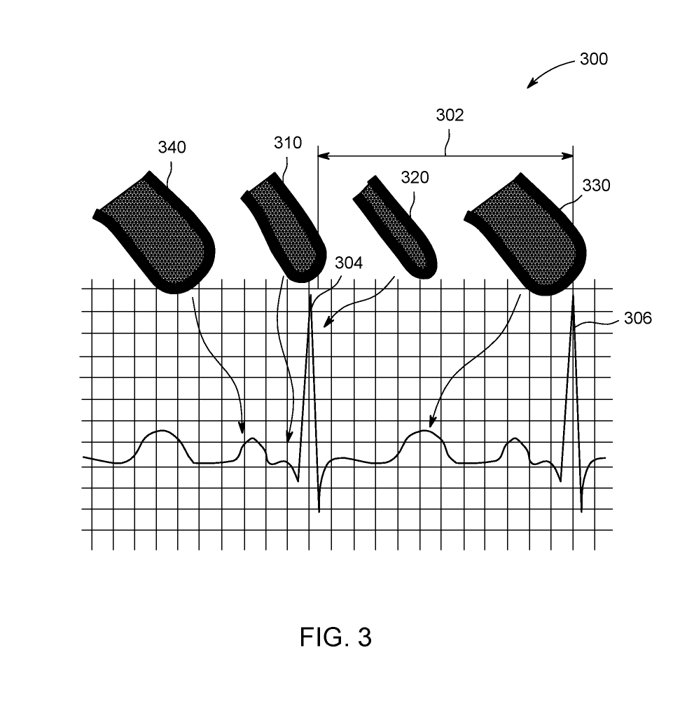

FIG. 3 illustrates an example cardiac wave 300 including associated left ventricle states schematically depicting corresponding volumes of the left ventricle at various portions of the cardiac cycle. As seen in FIG. 3, the cardiac wave 300 includes an R-R cycle 302 defined between first R point 304 and second R point 306. As seen in first left ventricle state 310 (occurring just before first R point 304) and second left ventricle state 320 (occurring just after first R point 304), the left ventricle volume is relative low close to the R portion of the cardiac wave 300. The left ventricle volume is substantially larger at third left ventricle state 330 (occurring subsequent to the R portion of the cardiac wave) and at the fourth left ventricle state 340 (occurring subsequent to the third left ventricle state 330) relative to the first left ventricle state 310 and second left ventricle state 320. In various embodiments, initial imaging information corresponding to one or both of the third left ventricle state 330 and fourth left ventricle state 340 is selected as the targeted initial imaging information, and initial imaging information corresponding to the first left ventricle state 310 and second left ventricle state 320 are not included with the target initial imaging information. For example, initial imaging information from a range of time or time window having endpoints defined by or corresponding to the third left ventricle state 330 and fourth left ventricle state 340 over a series of R-R cycles during the initial time range may be selected as the targeted initial imaging information.

With continued reference to FIG. 1, the depicted processing unit 120 is also configured to generate a blood pool activity curve using the targeted portions of the initial imaging information. For example, the blood pool activity curve may be used to derive diagnostic parameters (e.g., to determine calibration values). In some embodiments, a ratio of radiopharmaceutical uptake (e.g., as indicated by photon counts) in the blood to uptake in a muscle may be used in determining a calibration value to derive diagnostic parameters. It may be noted that the uptake values used for the blood and muscle (or other tissue) may come from different acquisition times. For example, the blood uptake may be determined for a blood pool (e.g., using targeted initial imaging information corresponding to phases of maximum volume of the left ventricle) for which imaging information has been acquired at an initial time, while the muscle (or other tissue) uptake may be determined using imaging information acquired at a subsequent time. The image may be reconstructed from imaging information acquired at the subsequent time using cardiac cycle stage information based on imaging information from the subsequent time as well as the initial time, or, as another example, may be reconstructed using imaging information acquired at both the subsequent and initial time.

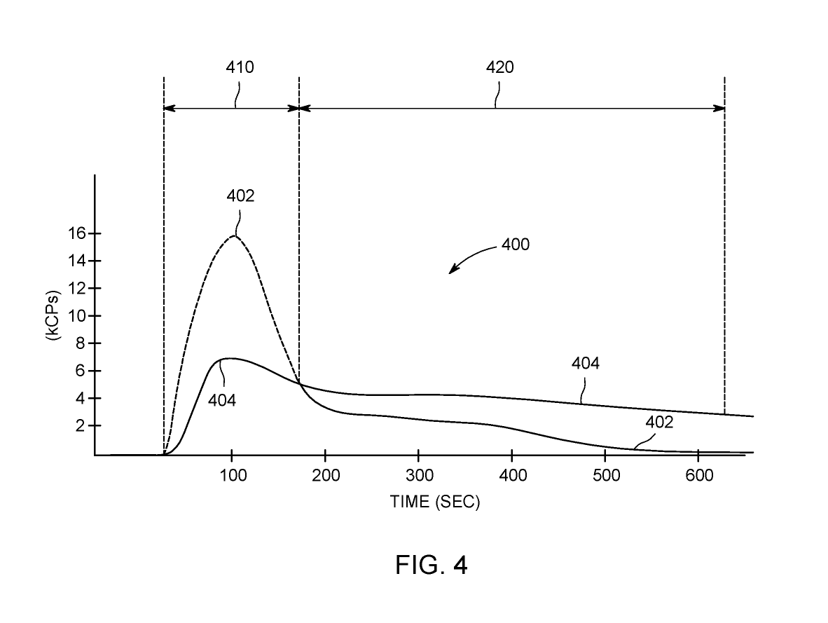

FIG. 4 depicts an example activity curve 400. As seen in FIG. 4, the activity curve 400 includes a blood portion 402 corresponding to radiopharmaceutical or tracer concentration in the blood, and a muscle potion 404 corresponding to uptake detected in association with the myocardium. Because the activity curve 400 has two different portions, the blood portion 402 may also be referred to a blood pool activity curve, and the muscle portion 404 may be referred to as a muscle activity curve (e.g., myocardium wall activity curve). Also, the activity curve 400 includes a first phase 410 and a second phase 420. The first phase 410 begins slightly after injection of a radiopharmaceutical, and may be referred to as a fast clearance phase. During the first phase 410 radiopharmaceutical or tracer passes through the myocardium for the first time, before uptake (or substantial or significant uptake) by the myocardium and/or other tissue (e.g., liver), and before dispersion within the entire blood pool of the patient. During the second phase 420, the radiopharmaceutical or tracer is evenly dispersed, with the second phase 420 corresponding to radiopharmaceutical or tracer uptake by the myocardium. In various embodiments, the initial time range may be located within the first phase 410, and the subsequent or general time range may be located within the second phase 420.

As noted herein, the imaging information during the initial time period of acquisition representing uptake by the blood in various embodiments is target imaging information including imaging information only from one or more portions of the cardiac cycle. Accordingly, the blood portion 402 of the activity curve 400 is represented as a dashed curve during the first phase 410, as not all portions of the imaging information are used as part of the targeted information used to determine uptake in the blood (e.g., during the initial time range). In various embodiments, by recording cardiac cycle information (e.g., ECG signal) alongside emission data, imaging information for a series of heartbeats may be segmented to a predefined number of segments, and, for blood pool curve determination input, only those segments most relevant to end diastolic volume may be utilized. Such segments may be used advantageously in blood pool curve determination, as the myocardium at such stages is at maximum volume, eliminating noise originating from other segments. Also, due to myocardium motion during the cardiac cycle, noise may be introduced into images. However, if only a selected segment (or segments) is used for sampling (during which the heart is theoretically static), motion artifacts may be reduced.

In some embodiments, information from different physical ranges or portions of the object (e.g., human patient) being imaged may be acquired during the initial time range relative to the general time range. For example, the processing unit may acquire initial imaging information for an initial time range over an initial field of view, and acquire general imaging information for a general time range over a general field of view, with the general field of view larger than the initial field of view. In various embodiments, additional detectors may be used to acquire the general field of view, and/or one or more detectors may be positioned and/or operated differently during the acquisition of the initial imaging information and the acquisition of the general imaging information.

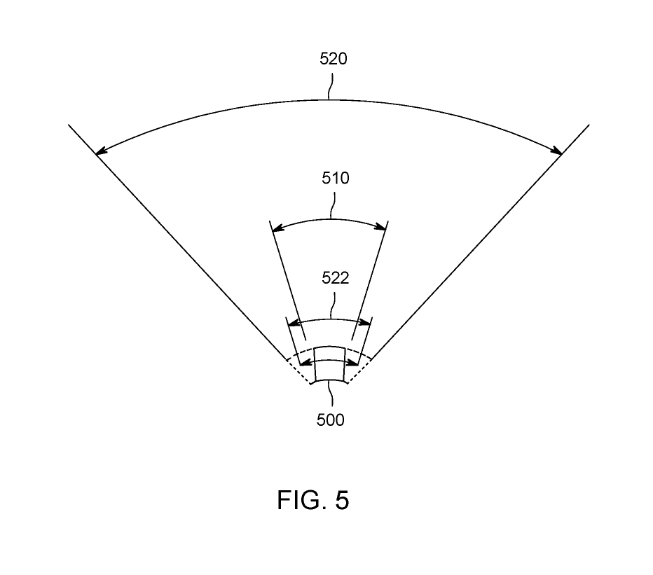

For example, in some embodiments, at least one detector may be swept (see, e.g., FIG. 2 and related discussion) while acquiring the general imaging information but not swept while acquiring the initial imaging information. Alternative, the at least one detector may be swept over a smaller range while acquiring the initial imaging information than while acquiring the general imaging information. FIG. 5 provides a schematic view of an example detector 500 having different fields of view for an initial imaging acquisition and a subsequent, general imaging acquisition. As seen in FIG. 5, the detector 500 has a first field of view 510 and a second field of view 520. In the illustrated embodiment, the first field of view 510 corresponds to a field of view of the detector 500 while not sweeping, and the second field of view 520 corresponds to a field of view of the detector 500 over a sweep range 522. The first field of view 510 may be employed during an initial acquisition over an initial time range, with the detector 500 swept to employ the second field of view 520 during a general acquisition over a general time range subsequent to the initial time range. For example, the first field of view 510 may be sized to include an imaging volume including the left ventricle, while the second field of view 520 is sized to include the entire heart and/or any surrounding tissue of interest. Accordingly, appropriate fields of view may be selected for different acquisitions to provide improved amounts of pertinent information for each acquisition and excluding imaging information that may not be used.

It may be noted that in various embodiments the imaging system 100 and/or processing unit 120 may perform operations alternatively or additionally to the use of a blood pool activity curve as discussed herein. For example, in various embodiments, the processing unit 120 is configured to acquire initial imaging information for an initial volume (e.g., a volume corresponding to an initial field of view as discussed herein, such as first field of view 510) over an initial time range. Further, the processing unit 120 may be configured to acquire general imaging information for a general volume (e.g., a volume corresponding to a general field of view as discussed herein, such as second field of view 520) over a general time range that is subsequent to the initial time range. The first volume is smaller than and, in some embodiments, included within the general volume. For example, the first volume may include a portion of the heart such as the left ventricle, and the second volume may include the entirety of the heart along with surrounding organs and/or other tissue. The processing unit 120 acquires the initial imaging information using different scan parameters than those used to acquire the general imaging information. As used herein, a scan parameter may be understood as a setting defining the operation of one or more detectors under the control of the processing unit 120 to acquire imaging information. Examples of scan parameters include, without limitation, sweep range, sweep speed, radial position, number of detector units to be used, scanning duration (in terms of time and/or total counts), and gantry rotational position. It may be noted that some of the scan parameters used to acquire the initial imaging information may be the same as some of the scan parameters used to acquire the general imaging information. The processing unit 120 is also configured to reconstruct an image using the initial imaging information and the general imaging information.

One or more scan parameters relating to sweep may vary between acquisition of the initial imaging information and the general imaging information in various embodiments. For example, a different sweep range and/or a different sweep speed may be used for acquiring the initial imaging information relative to the general imaging information. In some embodiments, the processing unit 120 sweeps at least one detector while acquiring the general imaging information but does not sweep at least one detector while acquiring the initial imaging information. In various embodiments, the same or different detectors may be used to acquire the initial imaging information as for the general imaging information.

In various embodiments the processing unit 120 includes processing circuitry configured to perform one or more tasks, functions, or steps discussed herein. It may be noted that “processing unit” as used herein is not intended to necessarily be limited to a single processor or computer. For example, the processing unit 120 may include multiple processors, FPGA's, ASIC's and/or computers, which may be integrated in a common housing or unit, or which may distributed among various units or housings (e.g., one or more aspects of the processing unit 120 may be disposed onboard one or more detector units, and one or more aspects of the processing unit 120 may be disposed in a separate physical unit or housing). It may be noted that operations performed by the processing unit 120 (e.g., operations corresponding to process flows or methods discussed herein, or aspects thereof) may be sufficiently complex that the operations may not be performed by a human being within a reasonable time period. For example, analyzing cardiac cycle information to determine portions of a cardiac cycle used for identifying targeted imaging information, determining blood pool curves, providing control signals to detector units, reconstructing an image, or the like may rely on or utilize computations that may not be completed by a person within a reasonable time period.

In the illustrated embodiment, the processing unit 120 includes a memory 130. Generally, the various aspects of the processing unit 120 act individually or cooperatively with other aspects to perform one or more aspects of the methods, steps, or processes discussed herein.

The memory 130 may include one or more computer readable storage media. The memory 130, for example, may store information describing previously determined boundaries of acquisition ranges, predetermined thresholds or other metrics utilized for determining boundaries of acquisition ranges, parameters to be utilized during performance of a scan (e.g., speed of rotation for acquisition range, speed of rotation for supplement zone, time or total count value over which an acquisition is to be performed), or the like. Further, the process flows and/or flowcharts discussed herein (or aspects thereof) may represent one or more sets of instructions that are stored in the memory 130 for direction of operations of the imaging system 100.

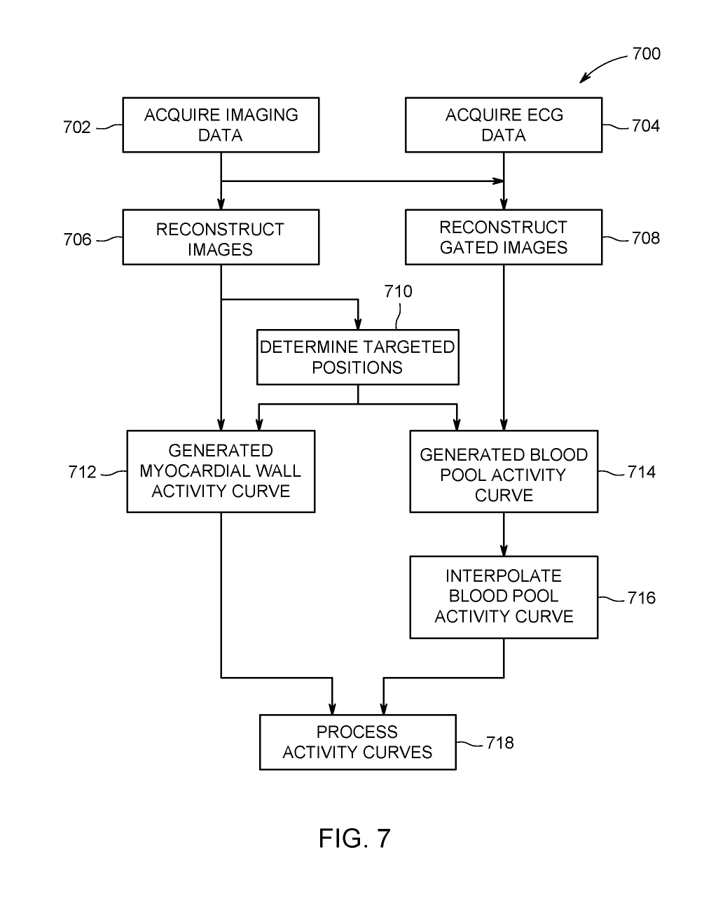

FIG. 7 provides a flowchart of a method 700 for imaging in accordance with various embodiments. The method 700 (or aspects thereof), for example, may employ or be performed by structures or aspects of various embodiments (e.g., systems and/or methods and/or process flows) discussed herein. In various embodiments, certain steps may be omitted or added, certain steps may be combined, certain steps may be performed concurrently, certain steps may be split into multiple steps, certain steps may be performed in a different order, or certain steps or series of steps may be re-performed in an iterative fashion. In various embodiments, portions, aspects, and/or variations of the method 700 may be able to be used as one or more algorithms to direct hardware (e.g., one or more aspects of the processing unit 120) to perform one or more operations described herein.

At 702, imaging data is acquired. In various embodiments, the imaging data (corresponding to detected events) is acquired and a saved as a list file, with each event time stamped. The imaging data is saved, allowing an operator to adjust temporal sampling specifications (e.g., frame duration) and energy sampling specifications (e.g., isotope and width of energy window). The imaging data in various embodiments includes initial imaging information as discussed herein. The imaging data may also include general imaging information as discussed herein.

At 704, ECG data is acquired. In various embodiments, the ECG data is acquired simultaneously with the imaging data acquired at 702, and synchronized to the list file discussed in connection with step 702. In the depicted embodiment, the ECG data is used, for example, for binning imaging data into bins synchronized to the cardiac cycle.

At 706, images are reconstructed. In various embodiments, detected events from the list file discussed in connection with step 702 are temporally reframed to a series of frames, for example according to a user defined reframing protocol. It may be noted that, in the illustrated embodiment, binning according to cardiac cycle is neglected for the images reconstructed at 706, with each of the frames reconstructed to create a corresponding “un-binned 3D image.”

At 708, gated images are reconstructed. In various embodiments, events from the list file are again temporally (e.g., as done in connection with step 706) and subsequently binned (e.g., based on the ECG data acquired at 704). For example, for each temporal frame, a bin reflecting or corresponding to the end diastolic cardiac phase may be used to reconstruct a gated image, with other bins disregarded when reconstructing the gated images. Each bin per frame may be reconstructed to create a corresponding “binned 3D image.” In the illustrated embodiment, a series of reconstructed binned reframed images are created.

At 710, targeted portions of the imaging information are determined. In the illustrated embodiment, images reconstructed at 706 are used to create targeted volumes of interest for a blood pool, and for a myocardial wall.

At 712, a myocardial wall activity curve is generated. In the illustrated embodiment, using the targeted myocardial wall volume of interest created at step 710, the reframed and reconstructed data of step 706 is sampled to create the myocardial wall activity curve (or time activity curve of the sampled myocardial wall). In various embodiments, the x coordinate in the curve is the time of the frame since the start of the acquisition, and the y coordinate in the curve is proportional to the activity within the targeted myocardial wall region in the “un-binned 3D image” of the corresponding frame.

At 714, a blood pool activity curve is generated. In the illustrated embodiment, the “binned 3D image” from 708 and the targeted blood pool volume of interest from 710 are used to create the blood pool activity curve (or time activity curve of the sampled blood pool. Accordingly, the blood pool activity curve may be generated using binned information while the myocardium wall activity curve is generated using non-binned data in some embodiments. The x coordinate of the blood pool activity curve in various embodiments is the time of the frame since the start of the acquisition, and the y coordinate is proportional to the activity within the targeted blood pool region in the “binned 3D image” (e.g., binned end diastolic 3D image) of the corresponding frame.

At 716 of the depicted embodiment, the blood pool activity curve is interpolated. In various embodiments, the blood pool activity curve is normalized to balance the reduced measured activity due to binning of each time frame. The interpolation or normalization may be performed to correct for the reduced total activity in each frame due to the process of discarding or disregarding a known number of bins, and/or to account for the fact that the total time in the end diastolic bins in the frames may be different (e.g., due to variations in heart rates.)

At 718, the activity curves are processed to generate or extract clinical data. For example, the blood pool activity curve and myocardium wall activity curve may be used to derive diagnostic parameters used in connection with image reconstruction.

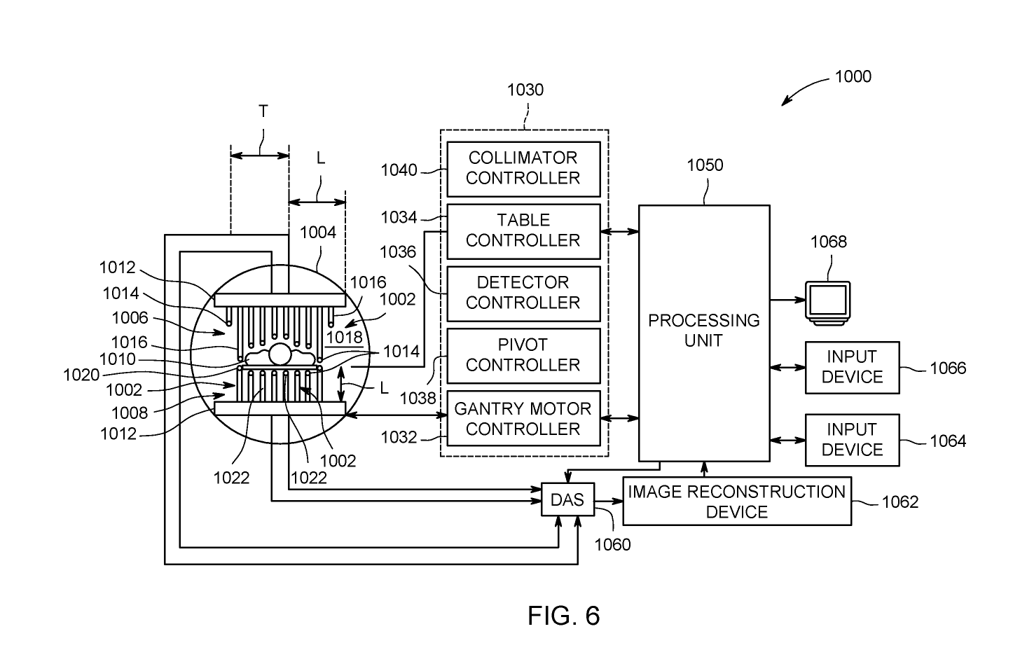

Embodiments described herein may be implemented in medical imaging systems, such as, for example, SPECT, SPECT-CT, PET and PET-CT. Various methods and/or systems (and/or aspects thereof) described herein may be implemented using a medical imaging system. For example, FIG. 6 is a schematic illustration of a NM imaging system 1000 having a plurality of imaging detector head assemblies mounted on a gantry (which may be mounted, for example, in rows, in an iris shape, or other configurations, such as a configuration in which the movable detector carriers 1016 are aligned radially toward the patient-body 1010). It should be noted that the arrangement of FIG. 6 is provided by way of example for illustrative purposes, and that other arrangements (e.g., detector arrangements) may be employed in various embodiments. In the illustrated example, a plurality of imaging detectors 1002 are mounted to a gantry 1004. In the illustrated embodiment, the imaging detectors 1002 are configured as two separate detector arrays 1006 and 1008 coupled to the gantry 1004 above and below a subject 1010 (e.g., a patient), as viewed in FIG. 6. The detector arrays 1006 and 1008 may be coupled directly to the gantry 1004, or may be coupled via support members 1012 to the gantry 1004 to allow movement of the entire arrays 1006 and/or 1008 relative to the gantry 1004 (e.g., transverse translating movement in the left or right direction as viewed by arrow T in FIG. 6). Additionally, each of the imaging detectors 1002 includes a detector unit 1014, at least some of which are mounted to a movable detector carrier 1016 (e.g., a support arm or actuator that may be driven by a motor to cause movement thereof) that extends from the gantry 1004. In some embodiments, the detector carriers 1016 allow movement of the detector units 1014 towards and away from the subject 1010, such as linearly. Thus, in the illustrated embodiment the detector arrays 1006 and 1008 are mounted in parallel above and below the subject 1010 and allow linear movement of the detector units 1014 in one direction (indicated by the arrow L), illustrated as perpendicular to the support member 1012 (that are coupled generally horizontally on the gantry 1004). However, other configurations and orientations are possible as described herein. It should be noted that the movable detector carrier 1016 may be any type of support that allows movement of the detector units 1014 relative to the support member 1012 and/or gantry 1004, which in various embodiments allows the detector units 1014 to move linearly towards and away from the support member 1012.

Each of the imaging detectors 1002 in various embodiments is smaller than a conventional whole body or general purpose imaging detector. A conventional imaging detector may be large enough to image most or all of a width of a patient's body at one time and may have a diameter or a larger dimension of approximately 50 cm or more. In contrast, each of the imaging detectors 1002 may include one or more detector units 1014 coupled to a respective detector carrier 1016 and having dimensions of, for example, 4 cm to 20 cm and may be formed of Cadmium Zinc Telluride (CZT) tiles or modules. For example, each of the detector units 1014 may be 8×8 cm in size and be composed of a plurality of CZT pixelated modules (not shown). For example, each module may be 4×4 cm in size and have 16×16=256 pixels (pixelated anodes). In some embodiments, each detector unit 1014 includes a plurality of modules, such as an array of 1×7 modules. However, different configurations and array sizes are contemplated including, for example, detector units 1014 having multiple rows of modules.

It should be understood that the imaging detectors 1002 may be different sizes and/or shapes with respect to each other, such as square, rectangular, circular or other shape. An actual field of view (FOV) of each of the imaging detectors 1002 may be directly proportional to the size and shape of the respective imaging detector.

The gantry 1004 may be formed with an aperture 1018 (e.g., opening or bore) therethrough as illustrated. A patient table 1020, such as a patient bed, is configured with a support mechanism (not shown) to support and carry the subject 1010 in one or more of a plurality of viewing positions within the aperture 1018 and relative to the imaging detectors 1002. Alternatively, the gantry 1004 may comprise a plurality of gantry segments (not shown), each of which may independently move a support member 1012 or one or more of the imaging detectors 1002.

The gantry 1004 may also be configured in other shapes, such as a “C”, “H” and “L”, for example, and may be rotatable about the subject 1010. For example, the gantry 1004 may be formed as a closed ring or circle, or as an open arc or arch which allows the subject 1010 to be easily accessed while imaging and facilitates loading and unloading of the subject 1010, as well as reducing claustrophobia in some subjects 1010.

Additional imaging detectors (not shown) may be positioned to form rows of detector arrays or an arc or ring around the subject 1010. By positioning multiple imaging detectors 1002 at multiple positions with respect to the subject 1010, such as along an imaging axis (e.g., head to toe direction of the subject 1010) image data specific for a larger FOV may be acquired more quickly.

Each of the imaging detectors 1002 has a radiation detection face, which is directed towards the subject 1010 or a region of interest within the subject.

The collimators 1022 (and detectors) in FIG. 6 are depicted for ease of illustration as single collimators in each detector head. Optionally, for embodiments employing one or more parallel-hole collimators, multi-bore collimators may be constructed to be registered with pixels of the detector units 1014, which in one embodiment are CZT detectors. However, other materials may be used. Registered collimation may improve spatial resolution by forcing photons going through one bore to be collected primarily by one pixel. Additionally, registered collimation may improve sensitivity and energy response of pixelated detectors as detector area near the edges of a pixel or in-between two adjacent pixels may have reduced sensitivity or decreased energy resolution or other performance degradation. Having collimator septa directly above the edges of pixels reduces the chance of a photon impinging at these degraded-performance locations, without decreasing the overall probability of a photon passing through the collimator.

A controller unit 1030 may control the movement and positioning of the patient table 1020, imaging detectors 1002 (which may be configured as one or more arms), gantry 1004 and/or the collimators 1022 (that move with the imaging detectors 1002 in various embodiments, being coupled thereto). A range of motion before or during an acquisition, or between different image acquisitions, is set to maintain the actual FOV of each of the imaging detectors 1002 directed, for example, towards or “aimed at” a particular area or region of the subject 1010 or along the entire subject 1010. The motion may be a combined or complex motion in multiple directions simultaneously, concurrently, or sequentially.

The controller unit 1030 may have a gantry motor controller 1032, table controller 1034, detector controller 1036, pivot controller 1038, and collimator controller 1040. The controllers 1030, 1032, 1034, 1036, 1038, 1040 may be automatically commanded by a processing unit 1050, manually controlled by an operator, or a combination thereof. The gantry motor controller 1032 may move the imaging detectors 1002 with respect to the subject 1010, for example, individually, in segments or subsets, or simultaneously in a fixed relationship to one another. For example, in some embodiments, the gantry controller 1032 may cause the imaging detectors 1002 and/or support members 1012 to move relative to or rotate about the subject 1010, which may include motion of less than or up to 180 degrees (or more).

The table controller 1034 may move the patient table 1020 to position the subject 1010 relative to the imaging detectors 1002. The patient table 1020 may be moved in up-down directions, in-out directions, and right-left directions, for example. The detector controller 1036 may control movement of each of the imaging detectors 1002 to move together as a group or individually. The detector controller 1036 also may control movement of the imaging detectors 1002 in some embodiments to move closer to and farther from a surface of the subject 1010, such as by controlling translating movement of the detector carriers 1016 linearly towards or away from the subject 1010 (e.g., sliding or telescoping movement). Optionally, the detector controller 1036 may control movement of the detector carriers 1016 to allow movement of the detector array 1006 or 1008. For example, the detector controller 1036 may control lateral movement of the detector carriers 1016 illustrated by the T arrow (and shown as left and right as viewed in FIG. 10). In various embodiments, the detector controller 1036 may control the detector carriers 1016 or the support members 1012 to move in different lateral directions. Detector controller 1036 may control the swiveling motion of detectors 1002 together with their collimators 1022. In some embodiments, detectors 1002 and collimators 1022 may swivel or rotate around an axis.

The pivot controller 1038 may control pivoting or rotating movement of the detector units 1014 at ends of the detector carriers 1016 and/or pivoting or rotating movement of the detector carrier 1016. For example, one or more of the detector units 1014 or detector carriers 1016 may be rotated about at least one axis to view the subject 1010 from a plurality of angular orientations to acquire, for example, 3D image data in a 3D SPECT or 3D imaging mode of operation. The collimator controller 1040 may adjust a position of an adjustable collimator, such as a collimator with adjustable strips (or vanes) or adjustable pinhole(s).

It should be noted that motion of one or more imaging detectors 1002 may be in directions other than strictly axially or radially, and motions in several motion directions may be used in various embodiment. Therefore, the term “motion controller” may be used to indicate a collective name for all motion controllers. It should be noted that the various controllers may be combined, for example, the detector controller 1036 and pivot controller 1038 may be combined to provide the different movements described herein.

Prior to acquiring an image of the subject 1010 or a portion of the subject 1010, the imaging detectors 1002, gantry 1004, patient table 1020 and/or collimators 1022 may be adjusted, such as to first or initial imaging positions, as well as subsequent imaging positions. The imaging detectors 1002 may each be positioned to image a portion of the subject 1010. Alternatively, for example in a case of a small size subject 1010, one or more of the imaging detectors 1002 may not be used to acquire data, such as the imaging detectors 1002 at ends of the detector arrays 1006 and 1008, which as illustrated in FIG. 6 are in a retracted position away from the subject 1010. Positioning may be accomplished manually by the operator and/or automatically, which may include using, for example, image information such as other images acquired before the current acquisition, such as by another imaging modality such as X-ray Computed Tomography (CT), MRI, X-Ray, PET or ultrasound. In some embodiments, the additional information for positioning, such as the other images, may be acquired by the same system, such as in a hybrid system (e.g., a SPECT/CT system). Additionally, the detector units 1014 may be configured to acquire non-NM data, such as x-ray CT data. In some embodiments, a multi-modality imaging system may be provided, for example, to allow performing NM or SPECT imaging, as well as x-ray CT imaging, which may include a dual-modality or gantry design as described in more detail herein.

After the imaging detectors 1002, gantry 1004, patient table 1020, and/or collimators 1022 are positioned, one or more images, such as three-dimensional (3D) SPECT images are acquired using one or more of the imaging detectors 1002, which may include using a combined motion that reduces or minimizes spacing between detector units 1014. The image data acquired by each imaging detector 1002 may be combined and reconstructed into a composite image or 3D images in various embodiments.

In one embodiment, at least one of detector arrays 1006 and/or 1008, gantry 1004, patient table 1020, and/or collimators 1022 are moved after being initially positioned, which includes individual movement of one or more of the detector units 1014 (e.g., combined lateral and pivoting movement) together with the swiveling motion of detectors 1002. For example, at least one of detector arrays 1006 and/or 1008 may be moved laterally while pivoted. Thus, in various embodiments, a plurality of small sized detectors, such as the detector units 1014 may be used for 3D imaging, such as when moving or sweeping the detector units 1014 in combination with other movements.

In various embodiments, a data acquisition system (DAS) 1060 receives electrical signal data produced by the imaging detectors 1002 and converts this data into digital signals for subsequent processing. However, in various embodiments, digital signals are generated by the imaging detectors 1002. An image reconstruction device 1062 (which may be a processing device or computer) and a data storage device 1064 may be provided in addition to the processing unit 1050. It should be noted that one or more functions related to one or more of data acquisition, motion control, data processing and image reconstruction may be accomplished through hardware, software and/or by shared processing resources, which may be located within or near the imaging system 1000, or may be located remotely. Additionally, a user input device 1066 may be provided to receive user inputs (e.g., control commands), as well as a display 1068 for displaying images. DAS 1060 receives the acquired images from detectors 1002 together with the corresponding lateral, vertical, rotational and swiveling coordinates of gantry 1004, support members 1012, detector units 1014, detector carriers 1016, and detectors 1002 for accurate reconstruction of an image including 3D images and their slices.

It should be noted that the particular arrangement of components (e.g., the number, types, placement, or the like) of the illustrated embodiments may be modified in various alternate embodiments, and/or one or more aspects of illustrated embodiments may be combined with one or more aspects of other illustrated embodiments. For example, in various embodiments, different numbers of a given module or unit may be employed, a different type or types of a given module or unit may be employed, a number of modules or units (or aspects thereof) may be combined, a given module or unit may be divided into plural modules (or sub-modules) or units (or sub-units), one or more aspects of one or more modules may be shared between modules, a given module or unit may be added, or a given module or unit may be omitted.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a processing unit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the term “computer,” “processor,” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer,” “processor,” or “module.”

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” may include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, the embodiments are by no means limiting and are exemplary embodiments. Many other embodiments will be apparent to those of skill in the art upon reviewing the above description. The scope of the various embodiments should, therefore, be determined with reference to the appended claims, along with the full scope of equivalents to which such claims are entitled.

In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

What is claimed is:

1. An imaging system comprising:
at least one detector unit configured to acquire nuclear medicine (NM) imaging information;
at least one processor operably coupled to the at least one detector unit, the at least one processor configured to:
acquire initial imaging information over an initial time range corresponding to administration of a radiopharmaceutical;
acquire cardiac cycle information over the initial time range;
determine targeted portions of the initial imaging information based on the cardiac cycle information to provide targeted initial imaging information;
generate an activity curve using the targeted portions of the initial imaging information; and
reconstruct an image using the activity curve.

2. The imaging system of claim 1, wherein the initial time range extends less than 200 seconds after administration of the radiopharmaceutical.

3. The imaging system of claim 1, wherein the at least one processor is configured to determine the target portions based on a portion of a cardiac cycle corresponding to dilation of a left ventricle.

4. The imaging system of claim 1, wherein the at least one processor is configured to determine the target portions based on a portion of a cardiac cycle corresponding to an end of diastole.

5. The imaging system of claim 1, wherein the at least one processor is configured to acquire general imaging information over a general time range subsequent to the initial time range, and reconstruct the image using the general imaging information.

6. The imaging system of claim 5, wherein the at least one processor is configured to acquire the initial imaging information over an initial field of view, and to acquire the general imaging information over a general field of view, wherein the general field of view is larger than the initial field of view.

7. The imaging system of claim 5, wherein the at least one processor is configured to sweep the at least one detector while acquiring the general imaging information but not while acquiring the initial imaging information.

8. A nuclear medicine (NM) multi-head imaging system comprising:
a gantry defining a bore configured to accept an object to be imaged;
plural detector units mounted to the gantry, each detector unit defining a corresponding view oriented toward a center of the bore, each detector unit configured to acquire imaging information over a sweep range corresponding to the corresponding view; and
at least one processor operably coupled to at least one of the detector units, the at least one processor configured to:
acquire initial imaging information, for at least one detector unit, for an initial volume over an initial time range;
acquire general imaging information, for at least some of the detector units, for a general volume over a general time range subsequent to the initial time range, wherein the initial volume is smaller than and included within the general volume, wherein the at least one processor is configured to use different scanning parameters for acquiring the initial imaging information than for the general imaging information; and
reconstruct an image using the initial imaging information and the general imaging information.

9. The system of claim 8, wherein the at least one processor is configured to sweep the at least one of the detectors while acquiring the general imaging information but not while acquiring the initial imaging information.

10. The system of claim 8, wherein the at least one processor is configured to:
acquire cardiac cycle information over the initial time range;
determine targeted portions of the initial imaging information based on the cardiac cycle information to provide targeted initial imaging information;
generate an activity curve using the targeted portions of the initial imaging information; and
reconstruct the image using the activity curve.

11. The system of claim 10, wherein the at least one processor is configured to determine the target portions based on a portion of a cardiac cycle corresponding to dilation of a left ventricle.

12. The system of claim 10, wherein the at least one processor is configured to determine the target portions based on a portion of a cardiac cycle corresponding to an end of diastole.

13. The system of claim 10, wherein the initial time range extends less than 200 seconds after administration of the radiopharmaceutical.

14. A method comprising:
acquiring initial imaging information with at least one of plural detector units mounted to the gantry, each detector unit defining a corresponding view oriented toward a center of the bore, each detector unit configured to acquire imaging information over a sweep range corresponding to the corresponding view for each detector unit, wherein the initial imaging information is acquired for an initial volume over an initial time range;
acquiring general imaging information, with at least some of the detector units, for a general volume over a general time range subsequent to the initial time range, wherein the initial volume is smaller than and included within the general volume, wherein different scanning parameters are used for acquiring the initial imaging information than for the general imaging information; and
reconstructing an image using the initial imaging information and the general imaging information.

15. The method of claim 14, wherein a different sweep range is used for acquiring the initial imaging information than for the general imaging information.

16. The method of claim 15, further comprising sweeping the at least one of the detectors while acquiring the general imaging information but not while acquiring the initial imaging information.

17. The method of claim 14, further comprising:
acquiring cardiac cycle information over the initial time range;
determining targeted portions of the initial imaging information based on the cardiac cycle information to provide targeted initial imaging information;
generating an activity curve using the targeted portions of the initial imaging information; and
reconstructing the image using the activity curve.

18. The method of claim 16, further comprising determining the target portions based on a portion of a cardiac cycle corresponding to dilation of a left ventricle.

19. The method of claim 16, further comprising determining the target portions based on a portion of a cardiac cycle corresponding to an end of diastole.

20. The method of claim 14, wherein the initial time range extends less than 200 seconds after administration of the radiopharmaceutical.
